(19) United States
(12) Patent Application Publication
(10) Pub. No.: US 2002/0128317 A1
(43) Pub. Date: Sep. 12, 2002

(54) Treatment of pathological conditions characterized by an increased IL-1 level

(75) Inventors: Suzy Charbit, Creteil (FR); Francois Schutze, St-Nom-La-Breteche (FR); Alain Taccoen, Le Chesnay (FR); Jean-Pierre Pellctier, Saint-Lambert (CA)

Correspondence Address: I.P. Docketing, PATERSON, BELKNAP, WEBB &TYLER, 1133 Avenue to the Americas, New York, NY, 10036, US

(73) Assignee: Laboratories NEGMA

(21) Appl. No.: 09/768,816

(22) Filed: Jan. 23, 2001

(51) Int. Cl. A61K 31/22; A61K 31/192

(52) U.S. Cl. 514/548; 514/569

(57) ABSTRACT

An enteral food composition for clinical or dietary use, comprises, in addition to carbohydrates and proteins or their hydrolysates the following components or their nutritional equivalents, per daily dosage: methionine (0.6-7 g), cysteine (0.5-2.5 g), folic acid (0.4-8 mg), pyridoxal (vitamin B 6) (3-20 mg), zinc (18-120 mg) and at least 400 kcal energy in the form of carbohydrates. These amounts are well above the Recommended Daily Allowance (RDA) values. Further preferred components include lecithin, cvanocobalamine, betaine and magnesium, as well as transsulfuration metabolites, ATP enhancers and antioxidants.

This invention relates to the treatment of pathological conditions characterized by an increased IL-1 level and to the use of diacterein and rhein in such treatment. The invention also relates to the treatment of pathological conditions in which inflammatory cytokines, such as interleukin-1 (IL-1) and/or tissue necrosis factor (TNF-α) are present and to the use of diacerein and rhein in their treatment. More particularly the invention relates to the use of diacerein and rhein compounds, which are relatively non-toxic, effective by oral ingestion and can be taken for long periods of time in the dosage required without side effects, in the treatment of the aforesaid conditions

The invention specifically resides in a method for treatment of pathological conditions characterized by an increased IL-1 and/or TNF-α level, such as rheumatoid arthritis, psoriatic arthritis, Wegener's disease, granulomatosis, asthma, pulmonary emphysema, Paget's disease, osteoporosis. bone metastases and atherosclerosis, by administering an effective amount of diacerein or rhein The invention also comprehends the treatment of certain conditions or disorders associated with the process of formation and development of the various types of blood cells and other formed elements by the hemopoietic tissues such as myeloma and myeloid leukemia.

The pathological conditions contemplated herein as benefiting from treatment with diacerein or rhein broadly encompass the inflammatory and autoimmune diseases.

In an earlier filed application, Ser. No. 09/663 528, filed Sep. 15, 2000, assigned to the same entity as the instant application, a method for treating osteoarthritis using diacerein as the active agent, alone or in combination with a nonsteroidal antiinflammatory drug (NSAID), has been disclosed Specifically, that application disclosed that although the cause of osteoarthritis is essentially unknown, it is likely that both the initiation and progression of the disease involve mechanical as well as biological events, resulting in the progressive degradation of the cartilage matrix associated with variable degrees of osteophytosis, subchondral bone sclerosis and synovial tissue alteration. The osteoarthritis affects all components of the joint, including bone, muscles, tendons, fibrous capsule, synovial membrane and articular cartilage. The cartilage destruction is believed to arise from an imbalance between chondrocyte-controlled anabolic and catabolic processes. Chondrocytes as well as synoviocytes maintain cartilage homeostasis and are activated to increase degradation of the cartilage matrix by inflammatory cytokines, such as interleukin-1 (IL-1) and tissue necrosis factor-α (TNF-α) which are released from the macrophages. Chondrocytes from osteoarthritis patients have a greater number of IL-1 receptors than cells from healthy individuals,

The pending application was, as already noted, directed to the method of treating osteoarthritis with diacerein, an anthraquinone having moderate antiinflammatory and analgesic activity, of the formula

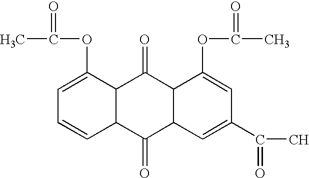

The inventors, in the course of their work with diacerein and osteoarthritis, also found that the mechanism of action of diacerein differs from that of other drugs used for the treatment of osteoarthritis, such as NSAIDs or corticosteroids. Diacerein, and more specifically its active diacetyl-derivative rhein, is an IL-1 inhibitor. The inventors demonstrated this in several studies in vitro and with animal models of osteoarthritis. The inventors found that neither diacerein nor rhein inhibit prostaglandin biosynthesis. In fact, no inhibitory effect was detected on the phospholipase, cyclooxygenase or lipooxygenase pathways. This unique mechanism of action of diacerein was thought to explain, at least in part, its efficacy in the symptomatic treatment of osteoarthritis. Furthermore, the absence of inhibition of prostaglandin biosynthesis was believed to explain the excellent gastric safety profile of diacerein during osteoarthritis treatment,

The administration of diacerein or rhein in the treatment of osteoarthritis derived from the unique mechanism of action of diacerein, wherein diacerein not only acted on the symptoms of the disease, inducing the short-term benefit for the patient of treating the pain and functional impairment, but in addition targeted the underlying pathologies, resulting in a long-term beneficial effect, Specifically, using the prescribed treatment protocol, it was found that diacerein had a significant effect on cartilage degradation and namely possessed activity for limiting the degradation of cartilage, and therefore for treating the course of the disease and its symptoms

The significance of the mechanism of action of diacerein as an inhibitor of IL-1 in connection with cartilage in osteoarthritis suggested to the inventors the invention herein, namely the use of diacerein and rhein in the treatment of inflammatory and autoimmune diseases including, without limitation, chronic heart failure, psoriatic arthritis, Wegener's disease, granulomatosis, endometriosis, asthma, Paget's disease, osteoporosis, bone metastasis, atherosclerosis, and hematopoietic disorders such as myeloma and myeloid leukemia.

The cytokines and lymphokines mediate the complex interactions involved in humoral and cellular immune responses, and participate in inflammation and local regulatory function, a notable example being the pyrogenic action of IL-1, which is mediated by the formation of prostaglandins. Several of the cytokines also appear to play an essential role in orchestrating the inflammatory process, especially interleukin 1 (IL-1) and tumor neurosis factor (TNF). Both IL-1 and TNF are derived from mononuclear cells and macrophages (as well as other cell types) and induce expression of numerous genes to promote the synthesis of a variety of proteins that contribute to inflammatory events. IL-1 and TNF are considered principal mediators of biological responses to bacterial lipopolysaccharides (endotoxins) and many other infectious stimuli. IL-1 and TNF appear to work in concert with each other and with growth factors (such as the granulocyte/macrophage colony stimulating factor, GM-CSF) and other cytokines, such as IL-8 and related chemotactic cytokines (chemokines), which can promote neutropil infiltration and activation.

IL-1 comprises two distinct polypeptides (IL-1αand IL-1β) that bind to the same receptor and produce similar biological responses. Plasma IL-1 levels are increased in patients with certain inflammatory processes (e.g., active rheumatoid arthritis). IL-1 can bind two types of receptors, an 80-kDa IL-1 receptor type 1 and a 68-kDa IL-1 receptor type 2, which are present on different types of cells,

TNF, originally termed “cachectin” because of its ability to produce a wasting syndrome, is comprised of two closely related proteins; mature TNF (TNFα) and lymphotoxin (TNFβ), both of which are recognized by the same cell surface receptor. There are two types of TNF receptors. a 75-kDa type 1 and a 55-kDa type 2 receptor for TNF.

IL-1 and TNF produce many of the same proinflammnatory responses, which include induction of fever, sleep, and anorexia; mobilization and activation of polymorphonuclear leukocytes; induction of cyclooxygenase and lipooxygenase enzymes; increase in adhesion molecule expression; activation of B-cells, T cells and natural killer cell; and stimulation of production of other cytokines. Other actions of these agents likely contribute to the fibrosis and tissue degeneration of the chronic proliferative phase of inflammation, stimulation of fibroblast proliferation, induction of collagenase, and activation of transcription factors, such as NFκ B and AP-1.

The NSAIDs are efficacious in providing symptomatic relief to subjects being treated for inflammatory and autoimmune diseases of the type set out above, but all available agents are associated with sometimes severe toxicity These agents have been highly useful for treatment of acute, self-limited inflammatory conditions. However, their ability to modify disease progression in chronic inflammatory settings is not well documented and remains an area of continuing controversy.

Advances in understanding the pathobiology of the inflammatory process has suggested several novel approaches for development of drugs to block this process. These include: (1) cytokine inhibitors (2) inhibitors of cell adhesion molecules, (b 3) phospholipase A₂ inhibitors, (4) inhibitors of lipooxygenase and leukotriene receptors, (5) isoform specific-inhibitors of cyclooxygenase, and now (6) diacerein.

Prior to the instant invention, diacerein and rhein were not utilized to modify the production or action of “proinflammatory” cytokines, such as IL-1 TNF, IL-6 and others,

An objective of the invention was to provide a method of treatment including the administration of diacerein or rhein to patients suffering from the inflammatory and autoimmune diseases, in which inflammatory cytokines such as interleukin-1 (IL-1) and tissue necrosis factor α (TNF-α) are present to an increased degree, as a result of which administration their disease would be limited, interrupted, enter into remission or be cured,

The over- or continued production of IL-1 and TNF leads to debilitation of normal host functions. The reduction of IL-1 and TNF synthesis or its effects is a major objective of the invention, namely to provide a therapy for the many diseases in which this condition exists. The agents for reducing the synthesis and/or for antagonizing the effects of IL-1 and TNF proposed herein are diacerein and rhein. The ability of these agents to block the effects of the cytokines has reduced the severity of the diseases in which an elevated production of cytokines (IL-1 and TNF) are present, particularly inflammatory or autoimmune diseases, and where the cytokine production is from neoplastic and leukemic cells.

In accordance with the invention, it was found that the administration of diacerein has an inhibitory effect on IL-1 and TNF-α levels in subjects having pathological conditions in which the levels of IL-1 and TNF-α are abnormally elevated or increased.

Pathological conditions characterized by an abnormally increased IL-1 and/or TNF-α levels include:

1) abnormal T and B lymphocyte activation, neutrophilia, neutrophil tissue infiltration, as well as increased lymphokine gene and receptor expression: the myeloproliferative syndromes: leukemias, lymphomas, myeloma and others.

2) endothelial cell activation, the pathology of the microcirculation: thrombosis, atherosclerosis Horton's arteritis and others.

3) eosinophil degranulation; the hyperimmune reactions allergy, asthma and others.

4) increased expression of adhesion molecules: [alcoholic cirrhosis chronic hepatitis B]

5) cyclooxygenase gene expression: rheumatoid arthritis

6) synthesis of collagenases and collagens; the hereditary. congenital and acquired diseases of collagen.

7) osteoblast activation: the diseases of the skeleton: osteoporosis, Paget's, osteoarthritis and others.

3) inflammatory bowel disease: Crohn's disease.

9) hematopoietic disorders: myeloma and myeloid leukemia and others

10) acute respiratory syndrome: asthma, pulmonary emphysema and others.

The treatment method of the invention is advantageously used in treating diseases such as rheumatoid arthritis, juvenile rheumatoid arthritis, chronic heart failure, psoriatic arthritis, psoriasis, osteoarthritis Wegener's disease, granulomatosis, endometriosis, bone metastases, atherosclerosis asthma. Paget's disease, osteoporosis, myeloma and myeloid leukemia. It has been found that the administration of effective amounts of diacerein is able to reduce signs and symptoms of these conditions and, most significantly, delay structural and other tissue damage and abnormalities in patients and inhibit the progression or course of the disease

Diacerein and rhein are known compounds and can be prepared by the processes described in U.S Pat. Nos. 6,057,461 and 5,948,924, and these patents are incorporated herein in their entirety by reference for the purpose of providing preferred methods of producing diacerein.

The diacerein as produced by those processes can be further purified to yield a product of the highest pharmaceutical usable purity using the process described in U.S. Pat. No 5,756,782, and therefore this patent is incorporated herein in its entirety by reference into this document.

As a general guide to required daily doses, the dosage of diacerein used is between about 25 mg and about 500 mg, depending on various factors, such as the type of disease, the patient's state and the like and is not dependent on body weight of the patient, at least in adults.

It is not convenient to use diacerein in aqueous solution, because it may not be sufficiently stable in water. Diacerein is virtually insoluble in water and in alcohol, this lack of solubility having to be taken into consideration in its administration. Preferably the diacerein or rhein is administered orally in the conventional solid unit dosage form.

The solid pharmaceutical composition may be in a pharmaceutical dosage form, which may desirably contain between 10 mg and 300 mg, preferably between 25 mg and 100 mg, of the diacerein or rhein. The pharmaceutical dosage form may conveniently be a tablet or pill or a capsule. Such compositions are prepared by conventional techniques, well known in the pharmaceutical art as reported e.g., in Remington's Pharmaceutical Science, 18th Ed., 1990.

The pharmaceutical composition may contain fillers or excipients, for example lactose, mannitol, sucrose, calcium sulphate, calcium phosphate and microcrystalline, cellulose; binders, for example tragacanth, acacia, starch and methylcellulose; disintegrants, for example corn starch and alginic acid; or lubricants, for example stearic acid and stearates and talc. Capsules are often of hard gelatin.

A particularly preferred pharmaceutical composition for oral administration is described in U.S. Pat. No. 5,952,383. This composition comprises the diacerein, a liquid support oil. a suspension agent, a homogenizing agent, a surfactant, and one or more pharmaceutically acceptable expedients or supports. These compositions are advantageously filled into soft or hard capsules, containing, for example, between about 20 mg and about 200 mg, preferably 50 mg of active principle per unit.

Another preferred pharmaceutical composition for oral administration is described in U.S. patent application 09/125,514, issued to U.S. Pat. No. ______. This composition is made by comicronization of the rhein or diacerein with sodium lauryl sulfate, the comicronized product in turn being formulated in various conventional application forms.

The entirety of the disclosures of these last two patents are incorporated herein by reference.

We claim;

1. Method of treating pathological conditions characterized by an increased IL-1 and/or TNF-α level which comprises administering to a subject having such condition a therapeutically effective amount of at least one member selected from the group consisting of diacerein and rhein in a pharmaceutical dosage form.

2. The method of claim 1 wherein said pathological condition is an inflammation or autoimmune disease.

3. The method of claim 1 wherein said condition is selected from the group consisting of rheumatoid arthritis, psoriatic arthritis Wegener's disease, granulomatosis, asthma, pulmonary emphysema, Paget's disease, osteoporosis, bone metastases, atherosclerosis, myeloma and myeloid leukemia.

4. The method of claim 1 wherein said group member is diacerein.

5. The method of claim 1 wherein said therapeutically effective amount is a daily dose of about 25 to about 500 mg of said group member,

6. The method of claim 1 wherein each unit dose of said pharmaceutical dosage form contains about 5 to about 500 mg of said group member.

7. The method of claim 1 wherein each unit dose of said pharmaceutical dosage form contains about 20 mg to about 200 mg of said group member.

8. The method of claim 1 wherein each unit dose of said pharmaceutical dosage form contains about 5 to about 100 mg of said group member.

9. The method of claim 1 where each unit dose of said pharmaceutical dosage form contains about 50 mg of said group member.

10. The method of claim 1 wherein said dosage form is a capsule.

11. Method of treating inflammatory and autoimmune conditions characterized by an increased IL-1 and or TNFα level which comprises modifying the production or action of proinflammatory cytokines including said IL-1 and/or TNFα by administering to a subject having such condition a therapeutically effective amount of at least one member selected from the group consisting of diacerein and rhein in a pharmaceutical dosage form.

12. The method of claim 11 wherein said group member is diacerein.

13. The method of claim 11 wherein said condition is rheumatoid arthritis.

14. Method for reducing the synthesis of IL-1 and TNFα which comprises administering to a subject a therapeutically effective amount of at least one member selected from the group consisting of diacerein and rhein in a pharmaceutical dosage form.
